(19) United States
(12) Patent Application Publication
(10) Pub. No.: US 2019/0053922 A1
(43) Pub. Date: Feb. 21, 2019

(54) NON-MIGRATING STENT DEVICES AND METHODS

(71) Applicant: WAKE FOREST UNIVERSITY HEALTH SCIENCES, Winston-Salem, NC (US)

(72) Inventors: Clifford Howard, JR., Winston-Salem, NC (US); Philip J. Brown, Winston-Salem, NC (US)

(21) Appl. No.: 16/079,813

(22) PCT Filed: Feb. 24, 2017
(86) PCT No.: PCT/US17/19389

§ 371 Date: Aug. 24, 2018

Related U.S. Application Data
(60) Provisional application No. 62/299,796, filed on Feb. 25, 2016.

(51) Int. Cl. A61F 2/848; A61F 2/915

(52) U.S. Cl. CPC A61F 2/848; A61F 2/915; A61F 2002/821; A61F 2002/91575; A61F 2002/8486

(57) ABSTRACT

Non-migrating stent devices and methods for using the devices are provided for treatment of visceral anastomosis strictures or vascular ostium high grade stenosis. For example, placement of a non-migrating stent may be appropriate following a surgical procedure such as hollow visceral anastomosis.

### RELATED APPLICATIONS

The present application claims priority to U.S. Provisional Patent Application No. 62/299,796, filed Feb. 25, 2016.

### FIELD

The disclosed invention relates to the medical field. Disclosed exemplary embodiments include non-migrating stent devices and methods for their use.

### BACKGROUND

Some medical procedures and anatomical arrangements present unique challenges with respect to stent design and placement. A variety of considerations may influence the choice of devices for management of a particular anatomical structure. In some cases, devices with special configurations may be optimal. For example, there is a high incidence of biliary enteric (˜10%), and ureter/ileal conduit (˜14%) post-surgical anastomosis strictures. This post-surgical complication is debilitating and inconvenient, often leads to prolonged hospital stays and multiple visits, and sometimes necessitates repeat or additional procedures.

Among the techniques currently used to address this problem are stricturoplasty, stent placement, long term drainage catheter use, and surgical/endoscopic ligation. Segmental stricturoplasty is successful approximately 66% of the time for treatment of biliary enteric strictures. The procedure often requires multiple dilatations over the span of several months. Treatment of visceral anastomosis strictures is often complicated by migration, bleeding and erosion. Biliary enteric and ileal conduit strictures require percutaneous access and drainage catheter placement which can be painful and require prophylactic maintenance exchange every 4-8 weeks. The drains are external and limit a patient's leisurely activities. Stents often migrate or occlude.

There is therefore a need for solutions that will obviate the practice of replacing external drains and the need for frequent prophylactic exchange of the drains in the setting of biliary enteric, ileal conduit, and colorectal strictures (e.g.), thus improving patient satisfaction and quality of life. In various medical circumstances the devices and methods described herein for treatment of visceral anastomosis strictures, or vascular ostium high grade stenosis, and the associated anatomical structures, can improve the post-surgical management and potential complications associated with various medical procedures.

### SUMMARY

Described herein are non-migrating stent devices and methods for their use. In some aspects, the invention includes a medical device configured to expand or fortify a stricture comprising a central conduit section, at least one anchoring section, and at least one transitional section positioned between the conduit section and the at least one anchoring section. In some embodiments, the device comprises two anchoring sections and two transitional sections, wherein each transitional section is positioned between the conduit section and one of the anchoring sections. In some embodiments, the conduit section, the at least one transitional section, and the at least one anchoring section are configured to form a substantially cylindrical structure having an interior lumen and a supportive exterior wall. In some embodiments, at least a portion of the supportive wall is configured as a mesh having openings.

In some embodiments of stent devices disclosed herein, at least one section of the device can assume at least two configurations: (i) a first configuration having a first outer diameter that is substantially constant along the longitudinal axis of the device, and wherein said first outer diameter allows the structure to be inserted in a stricture; and (ii) a second configuration wherein at least one section of the device has a second outer diameter that is larger than the first outer diameter. In some embodiments, all sections of the device can assume a second configuration. In some such embodiments, all sections of the device have a second outer diameter that is larger than the first outer diameter.

Some embodiments of non-migrating stent devices further include anchoring hooks. Such anchoring hooks may attach to the device at different longitudinal points or at a single point for each hook, or at multiple spots at the same longitudinal point. Some embodiments further include a living hinge, also called a flexure hinge. The living hinge can be an S-shaped hinge, an eyelet hinge, or a trough hinge, for example. In some stent device embodiments, deployment from a first configuration to a second configuration increases the minimum lumen diameter of the device. In some embodiments, deployment from a first configuration to a second configuration decreases the length of the device.

Some embodiments of the invention include methods for using stent devices. Methods for using a medical stent device to treat a stricture can include steps of placing the device into the stricture in a first configuration having a first outer diameter that is relatively constant along the longitudinal axis and that allows the structure to be inserted in the stricture, and deploying the device into a second configuration having a second outer diameter that is larger than the first outer diameter, so as to secure the device in place. In some aspects, deploying the device from a first configuration to a second configuration results in deployment of anchoring hooks positioned at the ends of the device. In some aspects, contact between the deployed anchoring hooks and the tissue associated with the stricture provides resistance so as to secure the device in the stricture. In some methods, the device is implanted in a visceral anastomosis stricture to expand or fortify the stricture. In other methods, the device is implanted in a vascular ostium high grade stenosis to expand or fortify the stenosis.

The illustrative examples provided with this disclosure are given to introduce the reader to the general subject matter discussed here and are not intended to limit the scope of the disclosed concepts. The following sections describe various additional features and examples with reference to the drawings in which like numerals indicate like elements, and directional descriptions are used to describe the illustrative examples but, like the illustrative examples, should not be used to limit the present disclosure.

### BRIEF DESCRIPTION OF FIGURES

FIG. 1: A depiction of typical biliary stricture anatomy is shown in FIGS. 1A-1B.

FIG. 2: An embodiment of a non-migrating stent device placed into a biliary stricture and deployed is shown in FIG. 2.

FIG. 3: FIGS. 3A-3C show one embodiment of a non-migrating stent device and provide an example of the movement of anchoring hooks during deployment from 3B to 3C.

FIG. 4: An embodiment of a non-migrating stent device is illustrated in a first configuration and a second (deployed) configuration in FIG. 4.

FIG. 5: Detail of the device illustrated in FIG. 4 is shown in FIG. 5.

FIG. 6: A minimalistic design embodiment of a non-migrating stent device, having broad “S” curves in transitional regions, is illustrated in FIG. 6.

FIG. 7: Detail of the device illustrated in FIG. 6, which features broad “S” curves in the transitional regions, is shown in FIG. 7.

FIG. 8: Another embodiment of a non-migrating stent device, featuring “S” hinges in the side struts of anchoring hooks, is pictured in FIG. 8.

FIG. 9: Another embodiment of a non-migrating stent device, featuring larger spaces between anchoring hooks, is pictured in FIG. 9.

FIG. 10: An embodiment of a non-migrating stent device featuring a larger initial diameter is shown in FIG. 10.

FIG. 11: An embodiment of a non-migrating stent device featuring 8 anchoring hooks is shown in FIG. 11.

FIG. 12: An embodiment of a non-migrating stent device featuring overall softer/smoother edges and curves is shown in FIG. 12.

FIG. 13: Detail of the device illustrated in FIG. 12, including eyelet hinges, is shown in FIG. 13.

FIG. 14: An embodiment of a non-migrating stent device featuring a flexible anchoring hook tip is illustrated in FIG. 14.

FIG. 15: Detail of the device illustrated in FIG. 14 is shown in FIG. 15.

FIG. 16: The flexible anchoring hook tip of the non-migrating stent device illustrated in FIGS. 14 and 15 is shown in FIG. 16.

FIG. 17: An embodiment of a non-migrating stent device featuring a modified anchoring hook tip having a double 4-bar linkage mechanism is shown in FIG. 17.

FIG. 18: Detail of the device illustrated in FIG. 17 is shown in FIG. 18.

FIG. 19: The anchoring hook tip having a double 4-bar linkage mechanism, of the non-migrating stent device illustrated in FIGS. 17 and 18, is shown in FIG. 19.

FIG. 20: A method embodiment for deployment of a non-migrating stent device via a balloon inflation advanced in 3 phases is illustrated in FIG. 20.

FIG. 21: A round eyelet hinge is illustrated in FIG. 21.

FIG. 22: An S-hinge is illustrated in FIG. 22.

FIG. 23: A slot eyelet hinge is illustrated in FIG. 23.

FIG. 24: A trough hinge is illustrated in FIG. 24.

### DEFINITIONS

As used in this specification, the singular forms “a,” “an,” and “the” include plural referents unless expressly and unequivocally limited to one referent.

The term “region(s)” is used interchangeably with the term “section(s)” throughout and refers to divisions along the lengthwise dimension of the devices disclosed herein.

The term “anchoring hooks” as used herein refers to device components designed to extend from the cylindrical plane of the device in a manner that allows the anchoring hooks to secure and maintain the placement of the device. A variety of designs for anchoring hooks are illustrated in the figures herein, and the anchoring hooks of any given end region (A or E, also denoted first or fifth region) may also vary in number. The term “hooks” is not intended to indicate sharpness, as in a fish hook, but rather conveys the capacity to provide traction or resistance to migration after placement and deployment of the anchoring hooks.

The term “longitudinal” (as in “longitudinal regions”) as used herein refers to the lengthwise aspects of the devices disclosed herein. For example, five longitudinal regions can represent five different lengthwise divisions or segments of a device.

The term “conduit section” or “conduit region” as used herein refers to the central or middle longitudinal section of the devices disclosed herein. The conduit section of the devices disclosed herein typically remains in a substantially cylindrical configuration whether the device is in a first configuration with a narrower conduit diameter or in a second (deployed) configuration with a broader conduit diameter. Alternatively the cylindrical shape of the conduit region may be flared near meeting with the transitional region when the device is deployed, or other structural adjustments may occur out-of-plane with the cylinder. Regardless of the exact shape, the conduit region serves to maintain an anatomical opening, such as a stricture, at a specific diameter.

The term “anchoring section” or “anchoring region” as used herein refers to the anchoring longitudinal section of the devices disclosed herein. The anchoring section can include anchoring hooks which deploy to secure the device in place via contact with tissues.

The term “anchoring hooks” as used herein refers to aspects of the devices disclosed herein that can change configuration to move outside of the cylindrical plane associated with a first configuration (i.e., undeployed) of the device to form a projection. For example, anchoring hooks can be attached to the anchoring section of a device via structural connections that allow and encourage the anchoring hooks to contact tissues so as to provide resistance to movement away from the site of placement and deployment.

The term “transitional” (as in “transitional region” or “transitional section”) as used herein refers to a connecting region between a middle cylindrical region of a stent device and an end region comprising anchoring hooks. Because the anchoring hooks can move out of the cylindrical plane in the change from a first configuration to a second configuration, a transitional region is designed to confer optimal flexibility to the end regions while also maintaining the strength of the device and the anchoring hooks. The transitional region provides a flexible coupling between the anchoring hooks and the center/middle conduit region of the stent. Typical uses will require the transitional region to expand asymmetrically to accommodate the deployed diameters of the anchoring hooks and center region. The transitional region will provide a smooth coupling, distributing and transmitting stress between both regions to reduce stress concentration, thereby reducing the possibility of stent clogging, stent degradation, and fatigue failure at the section interfaces/junctions and bridging the overall radial and longitudinal stiffness and support of the bordering hook and central regions.

The term “hinge connections” or “hinges” as used herein refers to structural aspects of the devices disclosed herein which can absorb large amounts of strain produced by the out-of-plane hinge motion that allows for deployment of the hooks. For example, in deployment, a hinge connection can allow greater range of movement for the structural aspects of the device that are directly attached to the hinge connection. A flexure hinge or living hinge is a hinge that comprises one solid piece that bends to create the hinging motion. Examples of flexure hinges (or living hinges) are eyelet hinges (e.g., round or slot), S-hinges, and trough hinges.

The term “deployed” (or deployment or other forms of the word deploy) as used herein refers to the structural change from a first configuration of a stent device to a second configuration of the device wherein anchoring hooks of the end regions demonstrate the most extreme change, such that they can provide traction against surrounding tissue and thereby prevent migration of the device away from the placement site.

The term “contact point” as used herein refers to the aspect of an anchoring hook that applies pressure to tissue surrounding a stricture or other anatomical structure to be stented. The contact point of an anchoring hook is designed to provide traction so as to resist allowing the migration of a stent device away from its original placement position.

The terms “expand” and “fortify” as used herein can refer to the diameter of a stricture being treated with the devices disclosed herein. For example, in some embodiments, a stent device can expand the diameter of a stricture. In some embodiments, a stent device can fortify the existing diameter of a stricture.

The term “lumen” as used herein refers to the interior aspects of a tube or cylinder. For example, the term lumen diameter refers to the diameter measured from the interior aspect of one wall of a stent device to the interior aspect of the opposite wall of the stent device, in portions configured as a tube or cylinder. A minimum lumen diameter is the interior diameter at the narrowest point along the cylinder. By contrast, the term “outer diameter” refers to the exterior aspects of the devices disclosed herein.

Unless otherwise specified, all diameters (e.g., sizes) herein refer to the internal diameter of a tube, in millimeters (mm). The commonly used catheter/surgical measure “French” is also provided in some instances for convenience, whereas one mm corresponds to 3 French.

### DETAILED DESCRIPTION

Post-surgical anastomosis strictures, such as biliary enteric, colorectal, and ureter/ileal conduit post-surgical anastomosis strictures, e.g., occur at a high rate following surgical procedures. Management of these strictures often presents a variety of challenges to medical personnel and to the patient. In some cases, a patient faces a long-term series of medical procedures, which presents additional challenges and complications. Anatomy and post-surgical recovery/status can vary considerably, even in patients of the same age or size, and a skilled healthcare provider, such as a surgeon, can appreciate that a particular patient's medical circumstances can influence the selection of non-migrating stent device in order to best accommodate a particular patient or stricture.

In some aspects, the invention described herein includes non-migrating stent devices and methods for using such devices to address a variety of visceral strictures (e.g., post-surgical anastomosis strictures).

### Devices

In some embodiments, a stent device may include a hollow cylindrical apparatus designed for short visceral anastomotic strictures and designed to prevent migration. For example, embodiments of a stent device can have five or more longitudinal regions (for example, regions A-E herein). For embodiments having five regions, all five regions can assume at least a first configuration and a second configuration.

Embodiments of non-migrating stent devices can assume at least two possible configurations. A first configuration of a non-migrating stent device can be substantially cylindrical and exhibit a relatively constant diameter along the length of the device. In a second configuration of a stent device, at least one region, and in certain embodiments, all regions can expand to increase the outer diameter of the device. For example, in embodiments having five longitudinal regions A-E, while the middle conduit region C can maintain a cylindrical shape as it expands, the other regions (A, B, D, and E) may expand outside of the cylindrical plane. In some embodiments, aspects of regions A, B, D, and E can also extend somewhat inside the cylindrical plane (i.e., slightly into the lumen of the device). At least one anchoring region (e.g., one or both of the first and fifth regions, A and E) can include anchoring hooks for securing a device once it has been placed and deployed. At least one transitional region (e.g., one or both of the second and fourth regions, B and D) can include transitional structures designed to accommodate a diameter expansion of cylindrical middle conduit region C, as well as any structural changes of the at least one anchoring region, and especially the deployment of anchoring hooks.

A non-migrating stent device may be constructed of any of a variety of biocompatible rigid or semi-rigid materials. Many suitable stent materials are known in the art. For example, stainless steel, cobalt chrome, other metal alloys, Nitinol (a known nickel titanium alloy), titanium, pyrrolitic carbon, plastics, or other materials may be suitable for constructing non-migrating stent devices. Woven or braided materials can also be appropriate. In some embodiments, a combination of different materials can be used to construct stent devices. For example, a coating of an additional biocompatible material may be applied to a stainless steel device. DACRON, woven velour, polyurethane, PTFE, ePTFE, heparin-coated fabrics, or bioengineered materials may be suitable for such coatings. A coating material may be applied to all exposed aspects or only some parts of a stent device. The materials suggested herein are examples only, and these lists of suggested options are non-limiting, as additional materials may be used to construct the described stent devices.

In some embodiments, a non-migrating stent device may comprise a therapeutic compound, so as to be a drug-eluting stent. For example, a non-migrating stent device may be coated with a slow-release drug formulation, such that the drug elutes from the stent slowly over an extended period of time after placement.

Upon selection of construction materials, various modes of construction known in the art can be employed. For example, laser cutting methods can be used to generate the non-migrating stent designs illustrated herein from a stainless steel or cobalt chrome cylinder. Similar laser cutting methods can be employed to generate non-migrating stent devices from other types of materials such as shape memory alloys. Further manufacturing details can be determined depending on the desired method of placement and deployment.

For example, a balloon deployable stainless steel stent device as described herein can be constructed. A balloon deployable stent device is designed for placement with a balloon inserted into the device lumen, such that inflation of the balloon causes deployment of the stent device from a first narrow configuration to a second broader configuration.

Alternatively, a shape memory alloy stent device can be constructed, expanded to the final desired configuration (a second configuration as described herein), and treated to set the shape (using heat, e.g.). A shape memory stent device can then be compressed into the first (original) narrower cylindrical configuration, enclosed by a sleeve to maintain the first configuration during placement, and then placed into the desired position (e.g., into a stricture) via known endoscopy or percutaneous methods. Once in place, removal of the sleeve allows the shape memory material to cause deployment from the first configuration to the second configuration (i.e., a self-expanding device). Those of skill in the art recognize that a self-expanding shape memory stent device is typically somewhat less rigid in a second, fully deployed configuration, but also easier to install than a balloon expandable stent device.

In some embodiments, non-migrating stent devices can include indicators, such as radio-opaque markings, to facilitate observation of the device by a healthcare provider via imaging. Such observation can be helpful during a procedure (e.g., to guide initial placement of the device) or subsequent to procedures (e.g., to verify at a later time that the device has remained in the desired position and location).

Thus, some embodiments of non-migrating stent devices may be configured to expand or fortify a stricture, wherein the device includes a central conduit section; at least one anchoring section; and at least one transitional section positioned between the conduit section and the at least one anchoring section. In some embodiments, the device comprises two anchoring sections and two transitional sections, wherein each transitional section is positioned between the conduit section and one of the anchoring sections. In some embodiments, the conduit section, the at least one transitional section, and the at least one anchoring section are configured to form a substantially cylindrical structure having an interior lumen and a supportive exterior wall. In some embodiments, at least a portion of the supportive wall is configured as a mesh having openings.

In some embodiments of stent devices disclosed herein, at least one section of the device can assume at least two configurations: (i) a first configuration having a first outer diameter that is substantially constant along the longitudinal axis of the device, and wherein said first outer diameter allows the structure to be inserted in a stricture; and (ii) a second configuration wherein at least one section of the device has a second outer diameter that is larger than the first outer diameter. In some embodiments, all sections of the device can assume a second configuration. In some such embodiments, all sections of the device have a second outer diameter that is larger than the first outer diameter.

Some embodiments of non-migrating stent devices further include anchoring hooks. Such anchoring hooks may attach to the device at different longitudinal points or at a single point for each hook, or at multiple spots at the same longitudinal point. Some embodiments further include a living hinge. The living hinge can be an S-hinge, an eyelet hinge, or a trough hinge, for example. In some stent device embodiments, deployment from a first configuration to a second configuration increases the minimum lumen diameter of the device. In some embodiments, deployment from a first configuration to a second configuration decreases the length of the device.

Some embodiments of the medical stent devices disclosed herein are designed for treatment of a stricture and can assume at least two configurations, including: a first substantially cylindrical configuration of the device, and a second configuration of the device wherein at least one region along the cylindrical length of the device is expanded from the initial cylindrical plane. In some embodiments, the stent devices can be configured to comprise a first, second, third, fourth and fifth longitudinal region, wherein: the first and fifth regions are positioned at the ends of the device and comprise anchoring hooks to function as the anchoring section; the third region is positioned in the middle of the device to function as the conduit section; and the second and fourth regions comprise transitional sections, wherein the second region is positioned between the first and third region, and the fourth region is positioned between the third and fifth region.

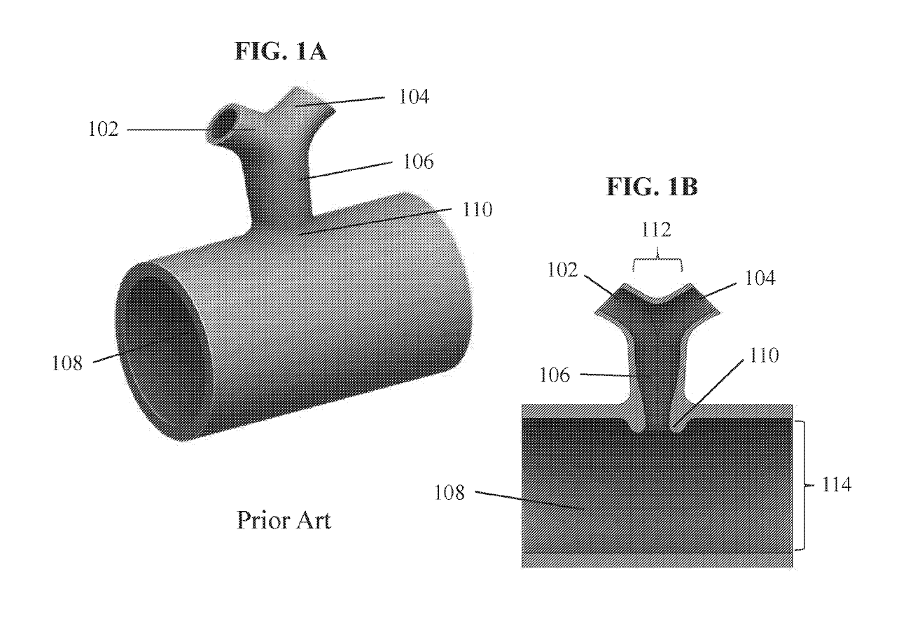

FIG. 1 shows two views of the typical human anatomical arrangement for the location of a biliary enteric post-surgical anastomosis stricture in FIGS. 1A and 1B. In one common site for biliary stricture, the common bile duct (CBD) 102 and the pancreatic duct 104 merge as they approach the duodenum 108. The major duodenal papilla 110 corresponds with the entry of the CBD 106 into the duodenum 108. The papilla 110 may be thickened by scar tissue as a component of the stricture. Typical dimensions, for example, are approximately 10 mm lumen diameter at the widest part of the CBD just above the papilla 112 and approximately 30 mm diameter in the lumen of the duodenum 114. A stricture can cause the CBD opening into the duodenum to be drastically narrowed, such that the narrowest aspect may be smaller than 8, 7, 6, 5, 4, 3, or 2 mm in diameter.

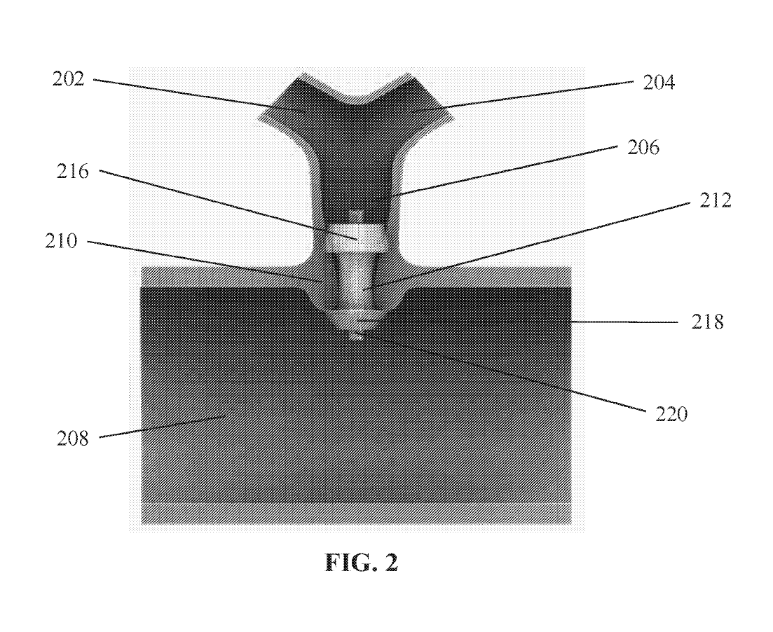

FIG. 2 shows an example of a deployed stent device embodiment placed in the biliary stricture. The common bile duct (CBD) 202 and the pancreatic duct 204 merge as they approach the duodenum 208. The major duodenal papilla 210 is a typical site for biliary enteric post-surgical anastomosis stricture, which may include scar tissue. An embodiment of a stent device 212 is placed into the CBD just above the stricture 206, such that one set of anchoring hooks 216 is within the CBD 206 while the set of anchoring hooks at the other end of the device 218 extends into the duodenum 208. The device is placed while in a first cylindrical configuration (not shown here), and when the device is in the proper position it is deployed into a second configuration (shown in FIG. 2), such that the middle conduit region of the device 212 expands to a larger cylindrical diameter, while the anchoring hooks 216 and 218 in the end regions of the stent device expand to secure the device in place. An additional implement 220 can be used to guide the stent device into the stricture, and can be removed after deployment of the stent device.

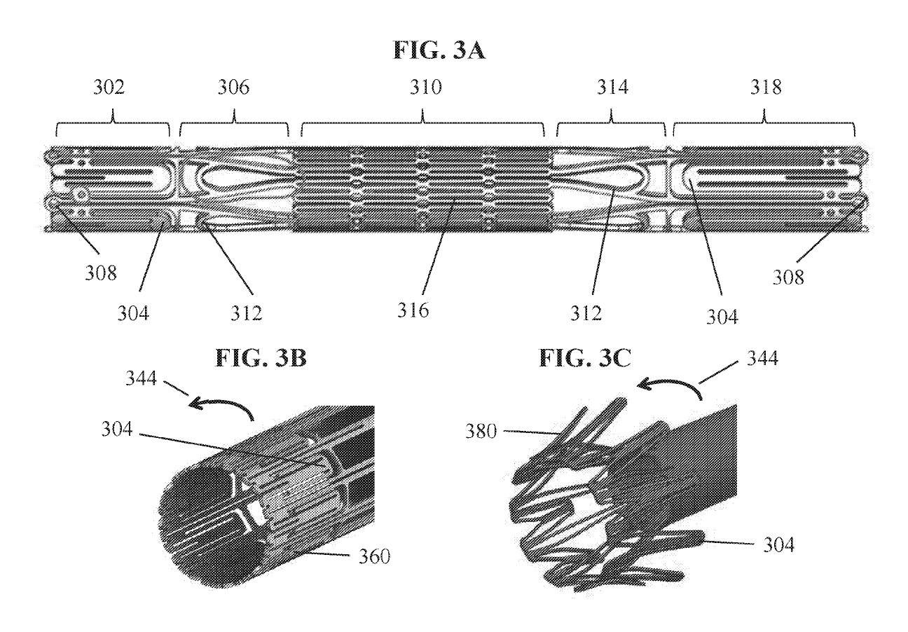

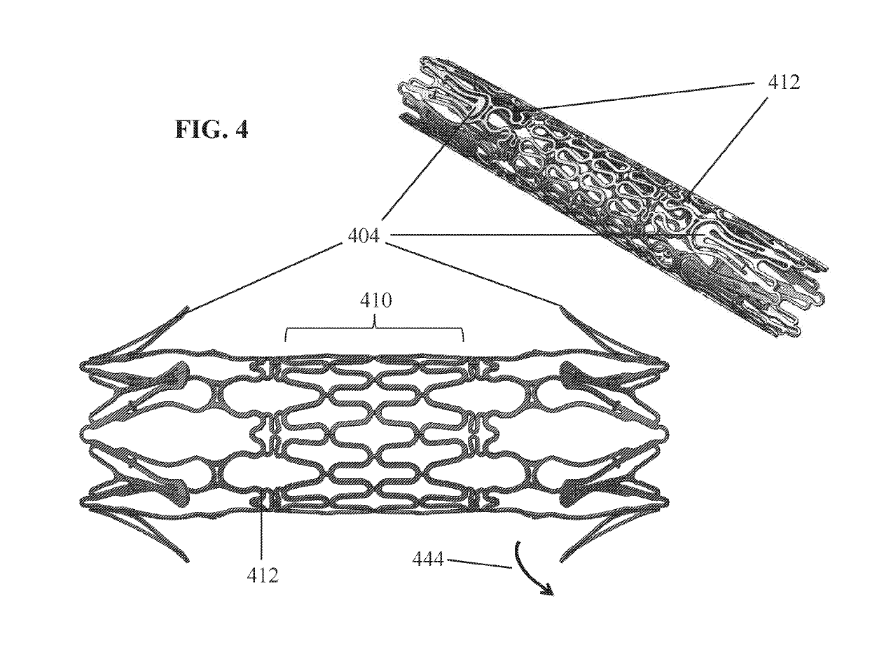

FIGS. 3A-3C: An embodiment of the stent device is illustrated in FIG. 3A. This example demonstrates five longitudinal regions A-E, also designated first 302, second 306, third 310, fourth 314, and fifth 318 regions. The first 302 and fifth 318 regions comprise anchoring hooks 304 and therefore function as anchoring regions of the stent. The second 306 and fourth 314 regions comprise transitional structures 312 to allow for increased expansion upon deployment from a first configuration of the device to a second configuration, and therefore function as transitional regions of the stent. The third 316 (middle or central) region functions as the conduit section and is designed to expand as the device is deployed, but can expand in a fashion that maintains a tubular or cylindrical plane in contrast with the other regions (e.g., see FIG. 4). In this embodiment, the first 302 and fifth 318 regions feature circular joints 308 between the anchoring hooks 304 to alleviate stress when the device is deployed or inflated. In one example, the device illustrated in FIG. 3A may comprise regions having lengths of 3.0 mm (first 302), 2.6 mm (second 306), 5.8 mm (third 310), 2.6 mm (fourth 314), and 4.4 mm (fifth 318). The broadest outer diameter in first and second configurations may be 8 mm and 10 mm in the central or middle longitudinal region of a non-migrating stent device, for example. Or other diameters and lengths may be used. The fully deployed outer diameter of a non-migrating stent device can be 2-16 mm, 3-15 mm, 4-14 mm, 5-13 mm, 6-12 mm, 7-11 mm, or 8-10 mm at the broadest point. Other sizes of device may be appropriate for some indications.

FIGS. 3B and 3C illustrate an example of deploying anchoring hooks, as in end regions A and E (i.e., first and fifth regions), showing a first configuration (FIG. 3B) of anchoring hooks 360 and a second configuration (FIG. 3C) of deployed anchoring hooks 380. In this example, the aspect of the hooks that contacts the tissue 304 deviates the most from the cylindrical plane upon deployment 380. The sets of anchoring hooks depicted in FIGS. 3A-3C differ slightly. For example, FIG. 3A illustrates rounded anchoring hooks 304 in regions A (first 302) and E (fifth 318), but the anchoring hooks of region A 302 are much shorter than those of region E 318. The anchoring hooks in FIGS. 3B and 3C 360 and 380 further differ from those illustrated in FIG. 3A regions A 302 and E 318, in that the contact points are more rounded in FIG. 3A as compared to the more squared contact points in the FIGS. 3B and 3C 360 and 380. The arrows 344 in FIGS. 3B and 3C illustrate the movement of anchoring hooks 360 and 380 as they are deployed.

FIG. 4 shows an embodiment of a non-migrating stent device. The first configuration is shown angled in the upper image, for comparison with the second deployed configuration shown as the lower horizontal image. Anchoring hooks 404 in the end regions (A and E) are shown in both images. Middle conduit region 410 is flanked by transitional regions 412. In the undeployed (angled) configuration, the anchoring hooks 404 are in the same cylindrical plane as the other regions, for a narrow first configuration. This allows guidance and insertion of the stent device into a stricture. In the deployed (horizontal) configuration, the anchoring hooks 404 extend outward, such that they provide traction against the stricture and thereby also provide resistance to forces or pressure that would otherwise cause the device to migrate away from the site of placement. The deployed configuration also has a wider diameter in the transitional regions 412 (B and D) as well as the cylindrical middle conduit region 410 (C). As a result of the deployment from the first configuration to the second configuration, the length of the device is also reduced. The design illustrated in FIG. 4 includes six (optionally, 4-8) anchoring hooks 404 at each end region. The arrow 444 shows the deployment motion of the anchoring hooks.

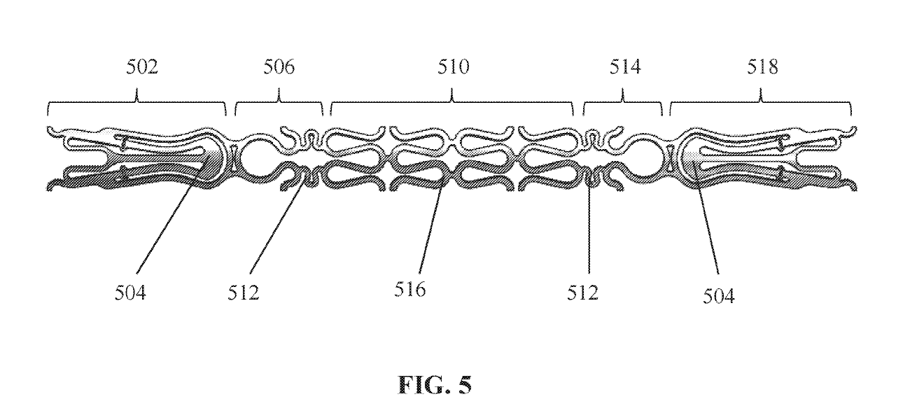

FIG. 5 shows detail of one section of an embodiment of the stent device illustrated in FIG. 4. The five longitudinal regions A-E are also designated first 502, second 506, third 510, fourth 514, and fifth 518 regions, respectively. The first 502 and fifth 518 anchoring regions comprise anchoring hooks 504. The second 506 and fourth 514 transitional regions comprise transitional structures 512 to allow for increased expansion upon deployment from a first configuration of the device to a second configuration. The third conduit 516 (middle) region is designed to expand in deployment, but can expand in a fashion that maintains a more tubular or cylindrical plane in contrast with the other regions (compare FIG. 4, e.g.). FIG. 5 shows one example of an open mesh structure that may be used as the wall of the stent.

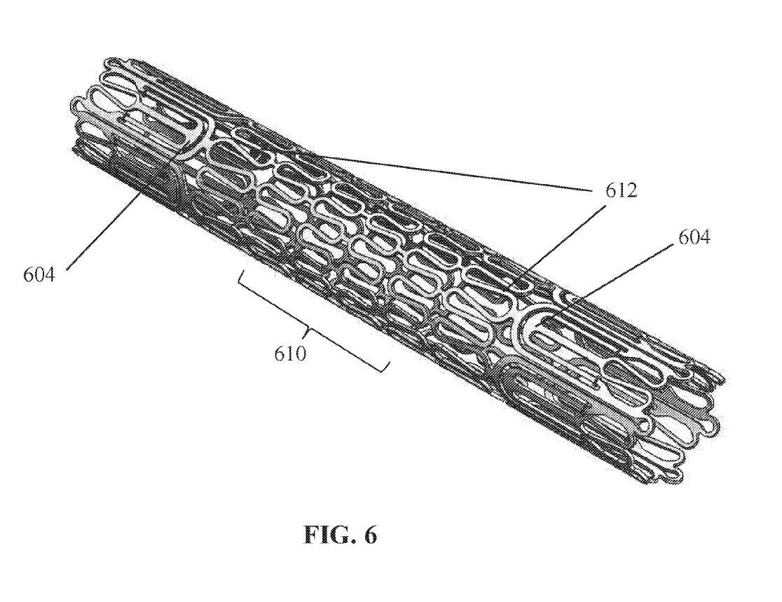

FIG. 6 shows another embodiment of a non-migrating stent device in a first configuration. Anchoring hooks 604 in the anchoring regions (A and E) are shown in the cylindrical plane (i.e., not deployed). Middle conduit region (C) 610 is flanked by transitional regions (B and D) 612. The design illustrated in FIG. 6 is simpler than some other designs illustrated herein. The transitional regions 612 of this embodiment are visually similar to the middle conduit region 610, but with broader “S” curves strategically placed to provide additional stability to a supporting structure for the anchoring hooks 604 in the end regions.

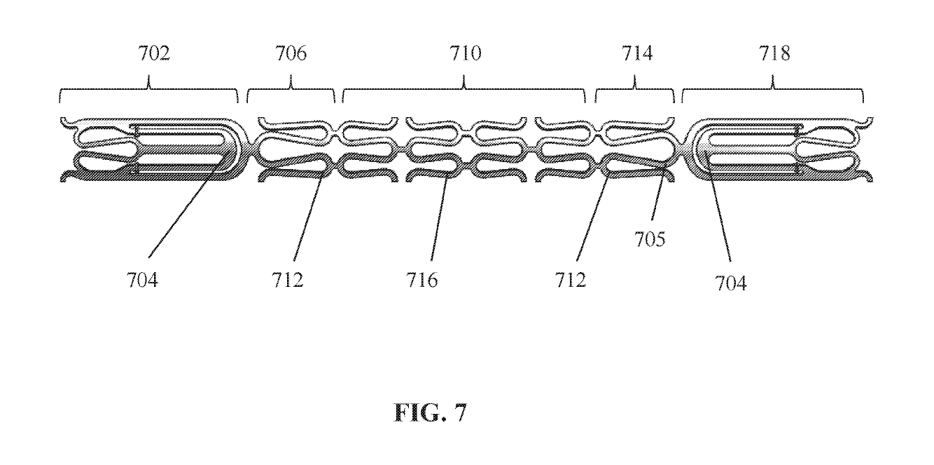

FIG. 7 shows detail of one section of the stent device embodiment illustrated in FIG. 6. The five longitudinal regions A-E are also designated first 702, second 706, third 710, fourth 714, and fifth 718 regions. The first 702 and fifth 718 anchoring regions comprise anchoring hooks 704. The second 706 and fourth 714 transitional regions comprise structures 712 to allow for increased expansion upon deployment from a first configuration of the device to a second configuration. The broader “S” curves 705 of the transitional regions 712 are strategically placed to provide additional stability to a supporting structure for the anchoring hooks 704 in the end regions. The third 716 (middle) region is designed to expand in deployment, but can expand in a fashion that maintains a more tubular or cylindrical plane in contrast with the other regions (compare FIG. 4, e.g.).

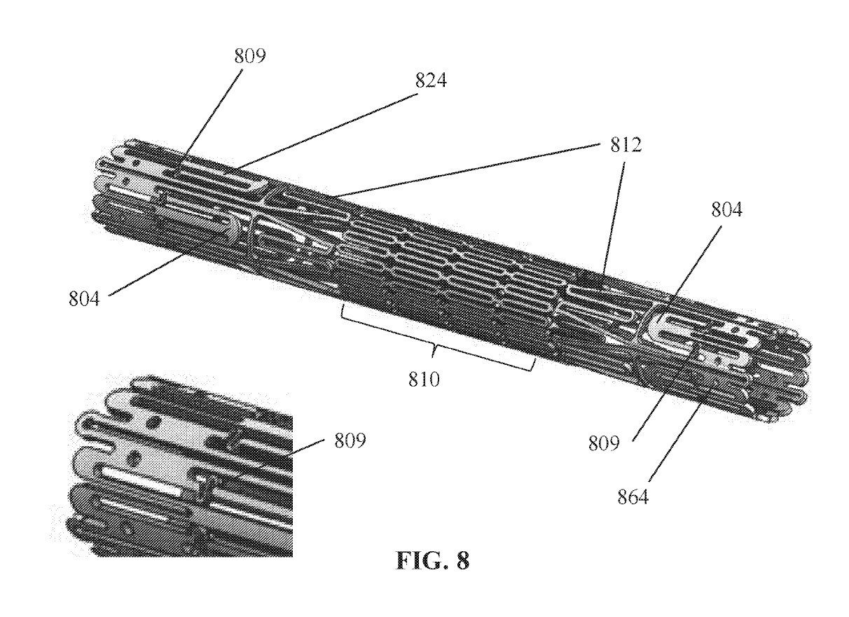

FIG. 8 shows another embodiment of a non-migrating stent device in a first configuration. Anchoring hooks 804 in the anchoring regions (first and fifth regions, or A and E) are shown in the cylindrical plane (i.e., not deployed). Middle conduit region (C) 810 is flanked by transitional regions (B and D) 812. This design features an “S” hinge 809 in the side struts of anchoring hooks 804. The inset at lower left provides a magnified view of this “S” hinge 809. This design is also asymmetrical, in that the first anchoring region A comprises longer anchoring hooks 824, while the fifth end region E comprises shorter anchoring hooks 864. In some embodiments, the short anchoring hooks 864 can be deployed inside a biliary duct, while the long anchoring hooks 824 can be deployed just outside the duct, in the duodenum.

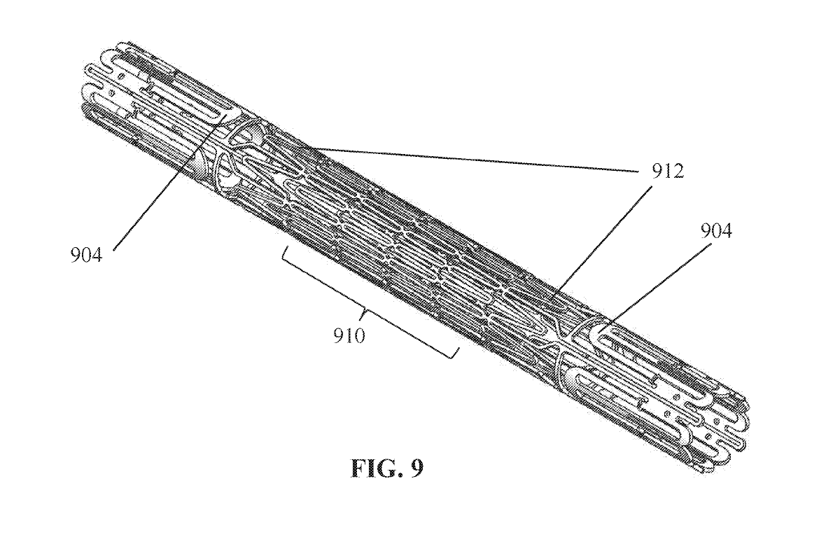

FIG. 9 shows another embodiment of a non-migrating stent device in a first configuration. The design illustrated here includes four anchoring hooks 904 in each of the anchoring regions (first and fifth regions, or A and E) to improve radial symmetry and allow easier simulation setup. The spaces between anchoring hooks are widened to reduce overall stiffness of the device. The anchoring hooks 904 are shown in the cylindrical plane (i.e., not deployed). Middle conduit region (C) 910 is flanked by transitional regions (B and D) 912. This transitional region design allows greater expansion and limits strain at maximum inflation/deployment diameter.

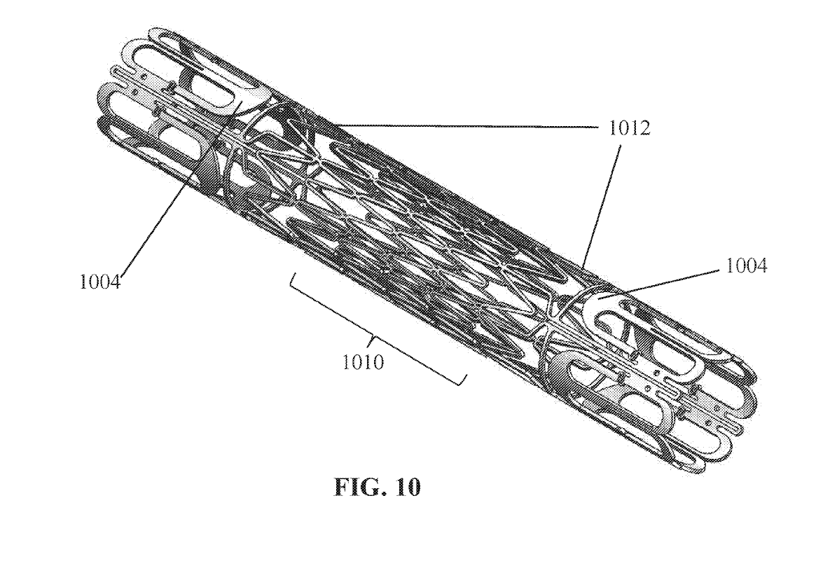

FIG. 10 shows another embodiment of a non-migrating stent device in a first configuration. This design features a larger initial diameter (i.e., first configuration diameter), such that the change in diameter from undeployed to deployed configurations may be smaller. Anchoring hooks 1004 in the end anchoring regions (first and fifth regions, or A and E) are shown in the cylindrical plane (i.e., not deployed). Middle conduit region (C) 1010 is flanked by transitional regions (B and D) 1012.

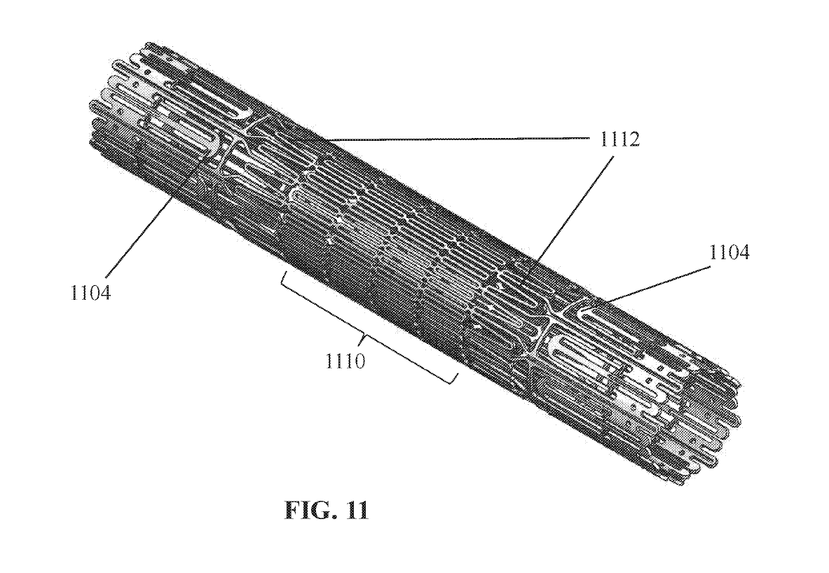

FIG. 11 shows another embodiment of a non-migrating stent device in a first configuration. The design illustrated here comprises 8 anchoring hooks 1104 in each of the end regions (first and fifth regions, or A and E). An increased number of anchoring hooks allows more contact points with the tissue surrounding a stricture, and thus provides more resistance to migration. The anchoring hooks 1104 are shown in the cylindrical plane (i.e., not deployed). Middle conduit region (C) 1110 is flanked by transitional regions (B and D) 1112.

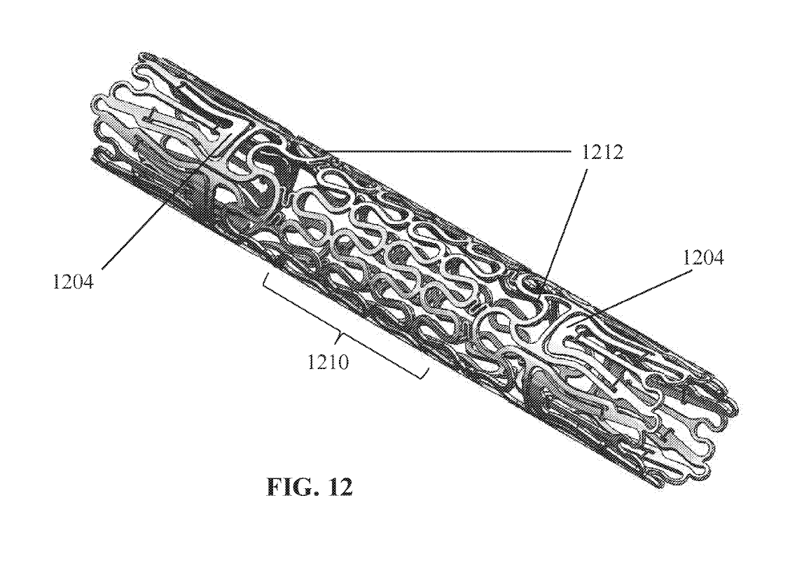

FIG. 12 shows another embodiment of a non-migrating stent device in a first configuration. Anchoring hooks 1204 in the end regions (first and fifth regions, or A and E) are shown in the cylindrical plane (i.e., not deployed) and have a more squared contact point for greater amount of contact with tissue surrounding the stricture. Middle conduit region (C) 1210 is flanked by transitional regions (B and D) 1212. The design illustrated in FIG. 12 features overall softer/smoother edges and curves to distribute stresses along curved features.

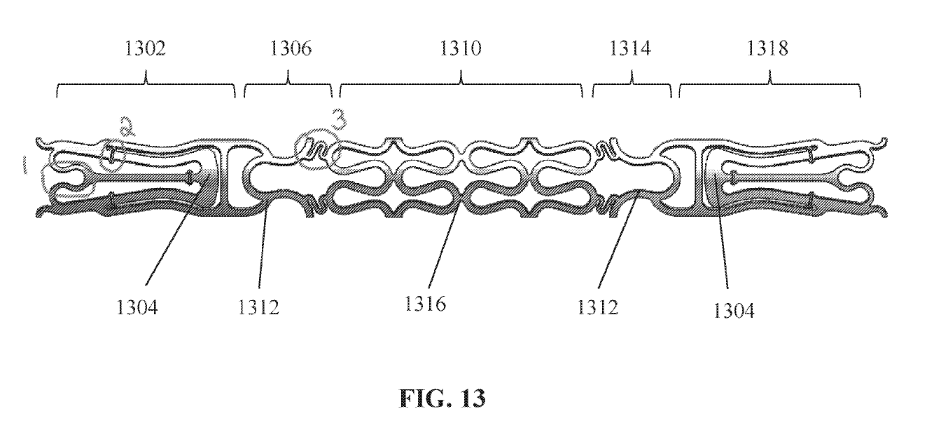

FIG. 13 shows detail of one section of the stent device embodiment illustrated in FIG. 12. The five longitudinal regions A-E are also designated first 1302, second 1306, third 1310, fourth 1314, and fifth 1318 regions. The first 1302 and fifth 1318 regions comprise anchoring hooks 1304. These anchoring hooks 1304 feature a less rounded tip (i.e., contact point) that allows for greater contact with the tissue surrounding a stricture. In the circled anchoring hook feature marked “1,” a curved mid-slot allows for increased stress relief. In the feature marked “2,” an eyelet hinge allows for more uniform stress relief and greater control and stability of the expanded joint. The second 1306 and fourth 1314 regions comprise transitional structures 1312 to allow for increased expansion upon deployment from a first configuration of the device to a second configuration. In the feature marked “3,” a linear expander (similar to S-hinge) can allow the transitional regions to compensate for foreshortening that may occur and reduce the amount of foreshortening. The third 1316 (middle) region is designed to expand in deployment, but can expand in a fashion that maintains a more tubular or cylindrical plane in contrast with the other regions (see, e.g., FIG. 4).

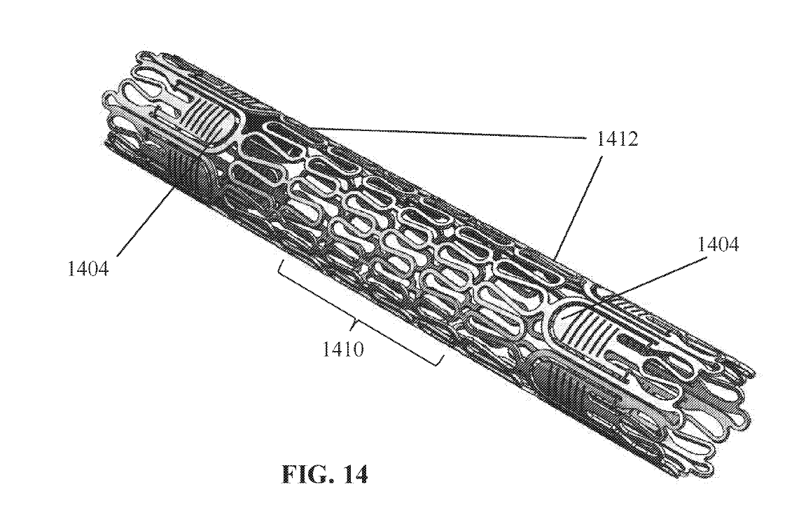

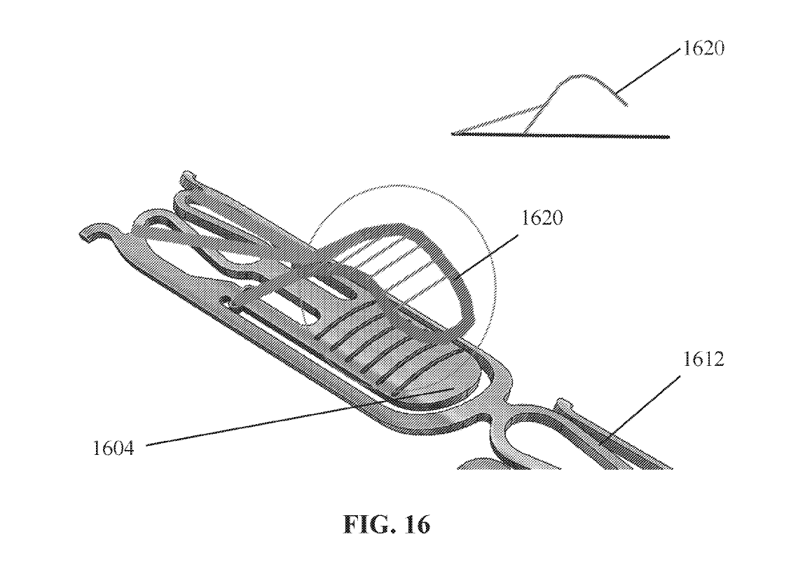

FIG. 14 shows another embodiment of a non-migrating stent device in a first configuration. Anchoring hooks 1404 in the end regions (A and E) are shown in the cylindrical plane (i.e., not deployed). This design features a modified anchoring hook tip, such that the hook tip is flexible as illustrated and described in FIG. 16. Middle conduit region (C) 1410 is flanked by transitional regions (B and D) 1412.

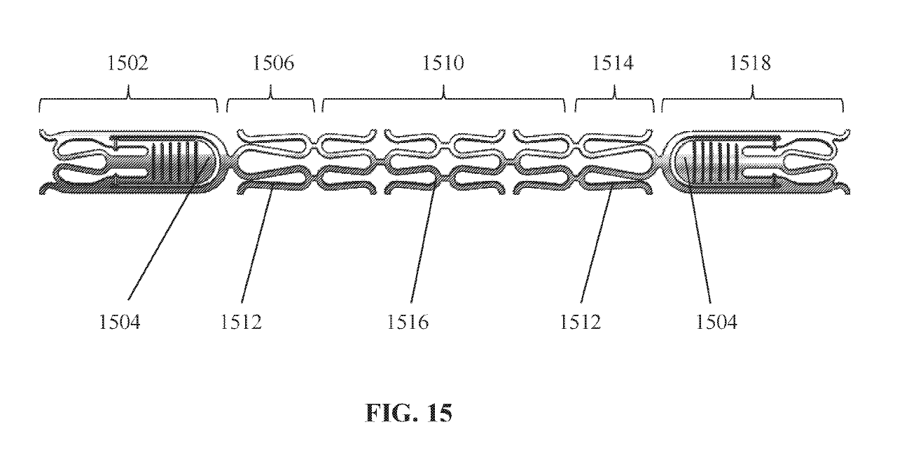

FIG. 15 shows detail of one section of the stent device embodiment illustrated in FIG. 14. The five longitudinal regions A-E are also designated first 1502, second 1506, third 1510, fourth 1514, and fifth 1518 regions. The first 1502 and fifth 1518 regions comprise anchoring hooks 1504. The second 1506 and fourth 1514 regions comprise transitional structures 1512 to allow for increased expansion upon deployment from a first configuration of the device to a second configuration. The third 1516 (middle) region is designed to expand in deployment, but can expand in a fashion that maintains a more tubular or cylindrical plane in contrast with the other regions (see, e.g., FIG. 4).

FIG. 16 shows a modified concept for anchoring hooks, based on the non-migrating stent device represented in FIGS. 14 and 15. Specifically, an aspect of an anchoring hook 1620 proximal to a contact point 1604 is designed to be flexible in the deployed configuration (showing tissue contact point of the anchoring hook deployed in the circled area), such that pressure applied by the contact point to a tissue surrounding a stricture, e.g., allows bending or curvature of the anchoring hook toward the lumen of the device. The transitional region 1612 provides additional flexibility while maintaining rigidity and strength. A side view of the curved flexible region 1620 of an anchoring hook is shown in the upper inset.

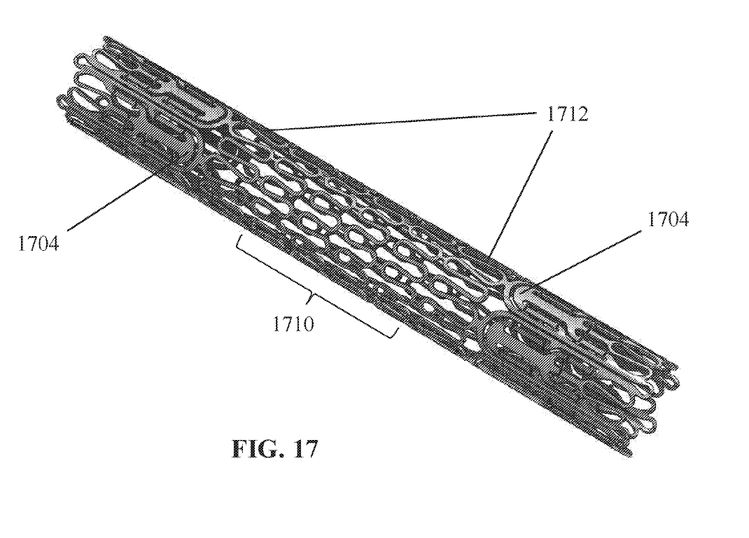

FIG. 17 shows another embodiment of a non-migrating stent device in a first configuration. Anchoring hooks 1704 in the end regions (A and E) are shown in the cylindrical plane (i.e., not deployed). This design features a modified anchoring hook tip, such that the hook tip has a double 4-bar linkage mechanism to increase radial force and reduce radial expansion. This hook mechanism is also compatible with other stent designs illustrated herein. Middle conduit region (C) 1710 is flanked by transitional regions (B and D) 1712.

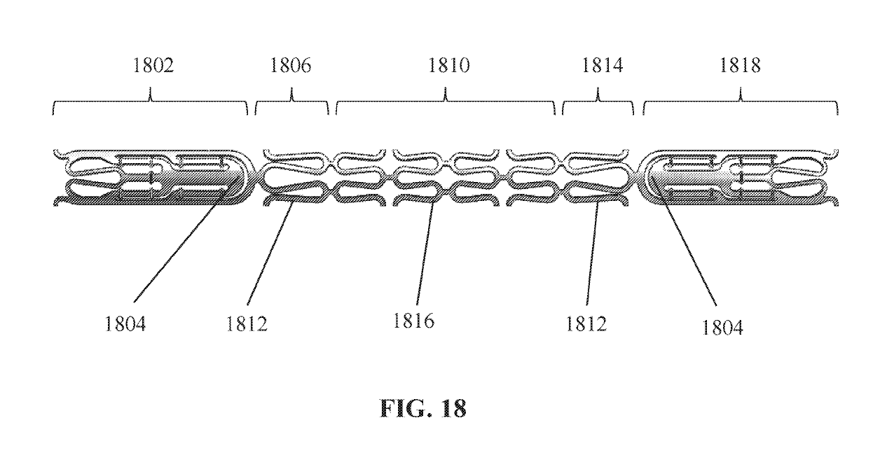

FIG. 18 shows detail of one section of the stent device embodiment illustrated in FIG. 17. The five longitudinal regions A-E are also designated first 1802, second 1806, third 1810, fourth 1814, and fifth 1818 regions. The first 1802 and fifth 1818 regions comprise anchoring hooks 1804. These anchoring hooks include a modified anchoring hook tip, such that the hook tip has a double 4-bar linkage mechanism to increase radial force and reduce radial expansion. This hook mechanism is also compatible with other stent designs illustrated herein. The second 1806 and fourth 1814 regions comprise transitional structures 1812 to allow for increased expansion upon deployment from a first configuration of the device to a second configuration. The third 1816 (middle) region is designed to expand in deployment, but can expand in a fashion that maintains a more tubular or cylindrical plane in contrast with the other regions (see, e.g., FIG. 4).

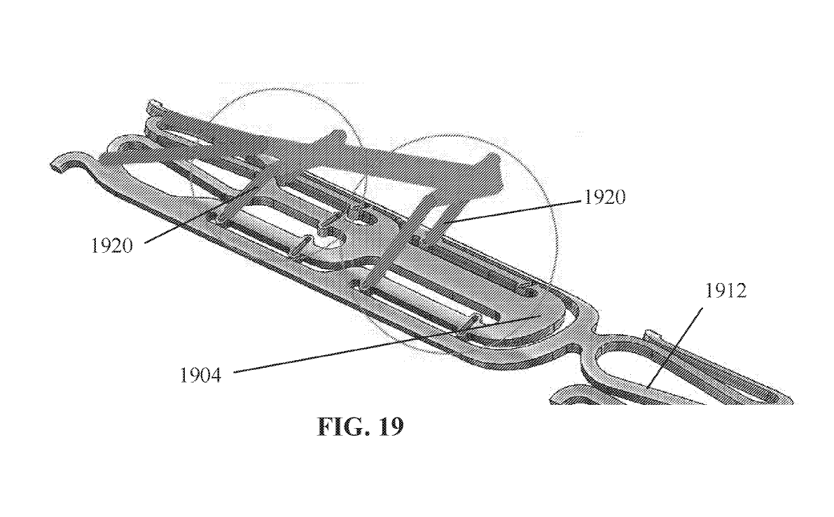

FIG. 19 illustrates a modified concept for anchoring hooks, based on the non-migrating stent device represented in FIGS. 17 and 18. Specifically, aspects of this anchoring hook design provide additional attachments 1920 to provide stability to the anchoring hook, increase radial force, and reduce radial expansion 1904. The additional attachments (showing deployed configuration in the circled areas), fortify the anchoring hook upon application of pressure via the contact point to a tissue surrounding a stricture, e.g. The transitional region 1912 provides additional flexibility while maintaining rigidity and strength.

### Methods

In some aspects, methods of using the stent devices described herein include analysis (e.g., via imaging technologies) wherein a healthcare provider determines that a particular anatomical structure, such as a post-surgical anastomosis stricture, is an appropriate target for treatment or management with a stent device. Following such a determination, a device size can be selected.

An appropriate size is selected, in part, based on outer diameter of a first and second configuration of the device. For example, a stent device must have a first configuration outer diameter small enough to facilitate insertion into a stricture or opening, but the device must also have a second configuration outer diameter large enough to apply pressure against the luminal wall of the target structure. Thus it is typical to “over size” the stricture when placing a stent device. As an example for visceral stent, for a common bile duct (CBD) anastomosis stricture, placement of a catheter no less than 12 French (4 mm) outer diameter, larger if possible, is desirable. The device should be large enough to apply sufficient pressure to the surrounding tissues such that migration away from the site of placement is inhibited. Thus in some embodiments, the stent device can expand the diameter of a stricture. In some embodiments, the stent device can fortify the existing diameter of a stricture.

In some embodiments, the stent devices described herein measure at least 6 mm outer diameter in the central or middle conduit region C, when fully deployed. The outer diameter at end regions A and E can measure up to 8 mm when fully deployed. This diameter corresponds to the size of metal stents typically placed in current practices; e.g., 8-10 mm metal stents are typically used for benign non-surgical strictures. For example, a non-migrating stent device diameter, when fully deployed, can be 2-16 mm, 3-15 mm, 4-14 mm, 5-13 mm, 6-12 mm, 7-11 mm, or 8-10 mm. Total length of the central portion of a non-migrating stent device can be between 1.6-3.4 cm, 1.8-3.3 cm, 2.0-3.0 cm, 2.2-2.8 cm, or 2.4-2.6 cm, and the length of a non-migrating stent device will shorten to varying degrees upon deployment to a second configuration, depending on the design of the stent device. However, the choice of stent device may vary in size based on anatomy and medical circumstances for the lumen in which it is deployed.

Additionally, the structural configuration of anchoring hooks can be an important consideration. For example, anchoring hooks can be symmetrical, both with respect to regions A and E and also within either region. Some stent device designs illustrated herein are symmetrical or nearly symmetrical (see, e.g., FIGS. 6-7 and 14-15). Alternatively, a stent device can include asymmetrical anchoring hooks, such that region A includes one anchoring hook design while region E employs a different design or different length of anchoring hooks (see, e.g., FIG. 8).

Stent devices can be placed using endoscopic procedures or percutaneous techniques as are known to those of skill in the art. The procedural and directional approach to the target anatomical structure may vary depending on the particular medical circumstances of a given patient, as is appreciated by the skilled healthcare provider (e.g., surgeon). Placement of a stent device into the target structure (e.g., stricture) can be achieved using devices and equipment known to those of skill in the art. For example, a catheter can be used to guide a stent device into the desired position. In many embodiments, a stent device is guided into the desired position within the target structure while in a first substantially cylindrical configuration. In many embodiments this first configuration also corresponds to the configuration of the stent device having narrowest outer diameter.

Once a device reaches the desired position, a healthcare provider can activate deployment of the device from a first configuration to a second configuration. A balloon can be used to facilitate deployment, for example as known in the art. By inflating a balloon within the lumen of a stent device, the device may be expanded from a first narrow lumen configuration to a second wide lumen configuration. In some embodiments a specialized balloon may be inflated within the device, such that some areas of the balloon expand sooner or to a greater extent than other areas of the balloon. For example, the wall of a balloon may be thicker or more resistant to inflation in some regions, leading to earlier inflation (and/or inflation to a greater extent) in other, thinner-walled regions of the balloon.

In some embodiments, as described in detail herein, a balloon inflation is advanced in phases. In some embodiments, non-migrating stent devices may deployed asymmetrically, such that anchoring hooks at one end of the device are deployed while anchoring hooks at the other end of the device remain undeployed. Or multiple (3 or more) configurations of the end regions (A and E) are possible, such that deployment can be partial at one or both ends.

For placement of shape memory stent devices, a sleeve surrounding the device may maintain the stent device in a compressed narrower cylindrical configuration during placement via known endoscopy or percutaneous methods. After the desired position is achieved, the sleeve can be retracted to allow the shape memory material to cause self-deployment from the first configuration to the second configuration (i.e., deployment of the self-expanding device). In some embodiments, a self-expanding stent device can be easier to install than a balloon expandable stent device.

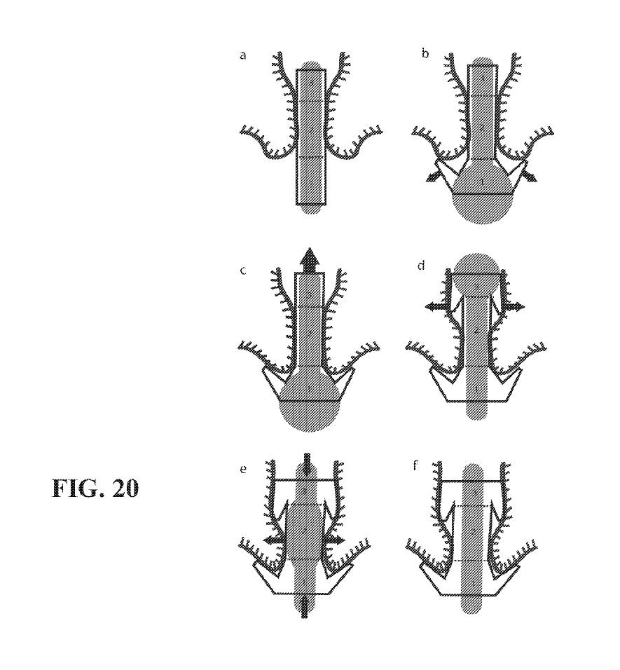

FIG. 20 shows an embodiment of a method for deploying a non-migrating stent device via balloon inflation advanced in three phases. The six panels of FIG. 20 are labeled (a)-(f). After the non-migrating stent device (with balloon in lumen) is inserted into a stricture and in the desired position, as shown in panel (a), the first segment of the balloon, protruding into the duodenum, is inflated, as shown in panel (b). This causes the deployment of anchoring hooks within the duodenum, as shown by arrows in panel (b). Next, as shown in panel (c), the stent device is drawn back into the common bile duct (CBD) to fully engage the deployed hooks on the duodenal end. In panel (d), the first segment of the balloon is deflated, while the third segment of the balloon is inflated, causing deployment of the anchoring hooks within the CBD. Next, in panel (e), the second segment of the balloon is inflated, while the third segment of the balloon is deflated, causing expansion and deployment of the middle conduit region of the non-migrating stent device, as shown by the arrows. The foreshortening of the stent device further engages the anchoring hooks at both ends and better secures the device in place, such that risk of migration is minimized. This example of a method for deployment of a non-migrating stent device is non-limiting, and other methods are also appropriate in some embodiments.

In some method embodiments, a single inflation phase is utilized for simple balloon insertion, as is known in the art of stent placement. In some embodiments, non-migrating stent devices may deployed asymmetrically, such that anchoring hooks at one end of the device are deployed while anchoring hooks at the other end of the device remain undeployed. Or deployment can be partial at one or both ends.

In other method embodiments, a shape memory stent device can be fixed (e.g., by heat treatment) in a fully deployed configuration, compressed into the first narrower cylindrical configuration, and enclosed by a sleeve to maintain the first configuration during placement. Once it has been placed into the desired position (e.g., into a stricture) via known endoscopy or percutaneous methods, retraction of the sleeve allows the shape memory material to cause deployment from the first configuration to the second configuration (i.e., a self-expanding device).

Thus, in some aspects, the invention disclosed herein includes a method for treating a stricture, comprising surgical placement of the device of any of the preceding claims into the stricture. In some embodiments, placement of the device comprises deploying the device from the first configuration to the second configuration. A method for using a medical stent device to treat a stricture can include steps of placing the device into the stricture in a first configuration having a first outer diameter that is relatively constant along the longitudinal axis and that allows the structure to be inserted in the stricture, and deploying the device into a second configuration having a second outer diameter that is larger than the first outer diameter, so as to secure the device in place. In some aspects, deploying the device from a first configuration to a second configuration results in deployment of anchoring hooks positioned at the ends of the device. In some aspects, contact between the deployed anchoring hooks and the tissue associated with the stricture provides resistance so as to secure the device in the stricture. In some methods, the device is implanted in a visceral anastomosis stricture to expand or fortify the stricture. In other methods, the device is implanted in a vascular ostium high grade stenosis to expand or fortify the stenosis.

The skilled medical professional will readily appreciate the range of additional medical circumstances in which visceral anastomosis strictures (e.g., as a result of post-surgical complications), may be addressed using the non-migrating stent devices, as described herein.

### Hinge Connections

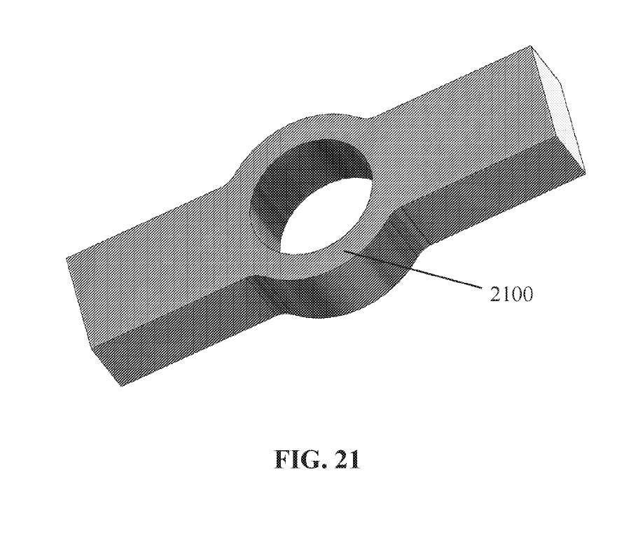

Some embodiments of stent devices comprise hinge connections, as are illustrated in many of the device figures. Hinge connections are designed to absorb large amounts of strain produced by the out-of-plane hinge motion that allows for change of configuration, deployment of anchoring hooks, and stability in the deployed configuration. FIGS. 21-24 show different designs for a flexure hinge or living hinge, so-called because the hinge comprises one solid piece that bends to create the hinging motion. Other functional hinges may be appropriate.

FIG. 21 shows an enlarged image of a round eyelet living hinge 2100.

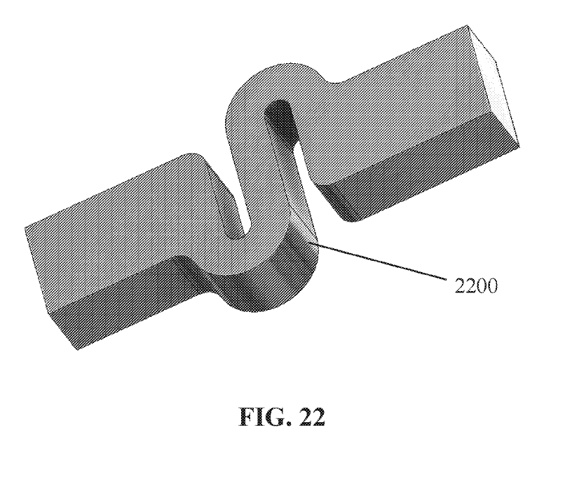

FIG. 22 shows an enlarged image of an S-shape living hinge 2200. FIGS. 8-11 illustrate stent devices that incorporate S-hinges, for example.

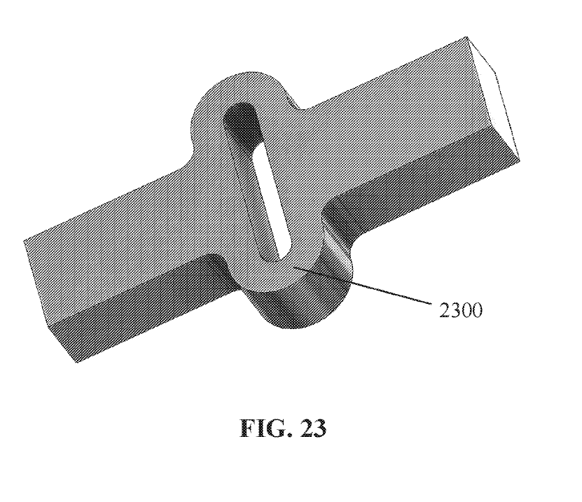

FIG. 23 shows an enlarged image of a slot eyelet living hinge 2300. FIGS. 4-7 and FIGS. 12-19 illustrate stent devices that incorporate slot eyelet living hinges, for example.

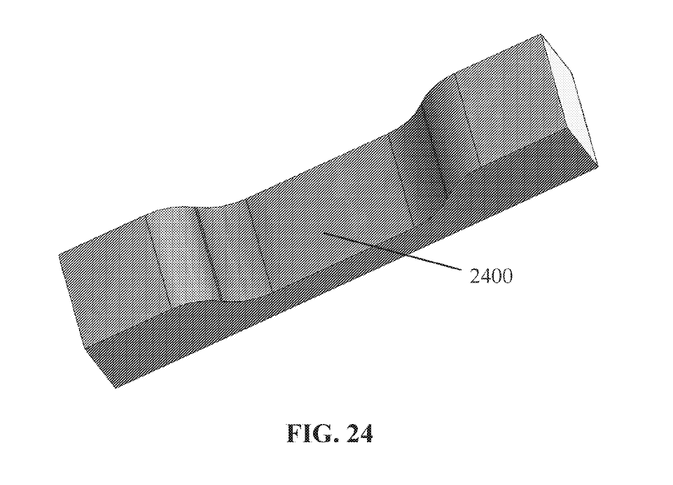

FIG. 24 shows an enlarged image of a trough living hinge 2400.

The non-migrating stent devices and methods for their use described herein can have further indications, such as for vascular ostium high grade stenosis. As will be appreciated by one of skill in the art, the examples of non-migrating stent devices and methods provided in the figures, as well as the examples for applications of the non-migrating stent devices and methods described below, are illustrative, and non-limiting, of the many possible embodiments of the present invention.

While the present invention has been disclosed with references to certain embodiments, numerous modifications, alterations and changes to the described embodiments are possible without departing from the scope and spirit of the present invention, as defined in the appended claims. Accordingly, it is intended that the present invention not be limited to the described embodiments, but that it have the full scope defined by the language of the following claims, and equivalents thereof.

Some of the embodiments of the technology described herein can be defined according to any of the following numbered paragraphs:

(1) A medical device configured to expand or fortify a stricture comprising a central conduit section; at least one anchoring section; and at least one transitional section positioned between the conduit section and the at least one anchoring section.

(2) The device of paragraph 1, wherein the device comprises two anchoring sections and two transitional sections, wherein each transitional section is positioned between the conduit section and one of the anchoring sections.

(3) The device of any of the preceding paragraphs, wherein each of the conduit section, the at least one transitional section, and the at least one anchoring section are configured to form a substantially cylindrical structure having an interior lumen and a supportive exterior wall.

(4) The device of paragraph 3, wherein at least a portion of the supportive exterior wall is configured as a mesh having openings.

(5) The device of any of the preceding paragraphs wherein at least one section of the device can assume at least two configurations: (i) a first configuration having a first outer diameter that is substantially constant along the longitudinal axis of the device, and wherein said first outer diameter allows the structure to be inserted in a stricture; and (ii) a second configuration wherein at least one section of the device has a second outer diameter that is larger than the first outer diameter.

(6) The device of any of the preceding paragraphs, further comprising anchoring hooks.

(7) The device of any of the preceding paragraphs, further comprising a living hinge.

(8) The device of paragraph 7, wherein the living hinge comprises an S-hinge, an eyelet hinge, or a trough hinge.

(9) The device of any of the preceding paragraphs, wherein deployment from a first configuration to a second configuration increases the minimum lumen diameter of the device.

(10) The device of any of the preceding paragraphs, wherein deployment from a first configuration to a second configuration decreases the length of the device.

(11) The device of any of the preceding paragraphs, wherein anchoring hooks comprise attachments to the device at different longitudinal points of the device.

(12) A medical device for treatment of a stricture, wherein said device can assume at least two configurations, comprising: a first substantially cylindrical configuration of the device, and a second configuration of the device wherein at least one region along the cylindrical length of the device is expanded from the initial cylindrical plane.

(13) The device of any of the preceding paragraphs configured to comprise a first, second, third, fourth and fifth longitudinal region, wherein: the first and fifth regions are positioned at the ends of the device and comprise anchoring hooks to function as the anchoring section; the third region is positioned in the middle of the device to function as the conduit section; and the second and fourth regions comprise transitional sections, wherein the second region is positioned between the first and third region, and the fourth region is positioned between the third and fifth region.

(14) A method for treating a stricture, comprising surgical placement of the device of any of the preceding paragraphs into the stricture.

(15) The method of paragraph 14, wherein placement of the device comprises deploying the device from the first configuration to the second configuration.

(16) A method for using a medical stent device to treat a stricture, comprising: placing the device into the stricture in a first configuration having a first outer diameter that is relatively constant along the longitudinal axis and that allows the structure to be inserted in the stricture; and deploying the device into a second configuration having a second outer diameter that is larger than the first outer diameter, so as to secure the device in place.

(17) The method of any of paragraphs 14-16, wherein deploying the device from a first configuration to a second configuration results in deployment of anchoring hooks positioned at the ends of the device.

(18) The method of any of paragraphs 14-17, wherein contact between the deployed anchoring hooks and the tissue associated with the stricture provides resistance so as to secure the device in the stricture.

(19) The method of any of paragraphs 14-18 wherein the device is implanted in a visceral anastomosis stricture to expand or fortify the stricture.

(20) The method of any of paragraphs 14-19 wherein the device is implanted in a vascular ostium high grade stenosis to expand or fortify the stenosis.

### EXAMPLES

The descriptions of certain examples, including illustrated and described examples, are presented only for the purpose of illustration and description and are not intended to be exhaustive or to limit the disclosure to the precise forms disclosed. Numerous modifications, adaptations, and uses thereof will be apparent to those skilled in the art without departing from the scope of the disclosure.

### Example 1: Biliary Enteric Anastomosis Stricture

Pancreatoduodenectomy (PD) is used in the surgical treatment of biliary and pancreatic disorders. Anastomotic stricture is a major complication. The current standard of care treatment for PD anastomotic stricture includes percutaneous transhepatic cholangiogram (PTC) and percutaneous biliary drain (PBD) placement with serial drain exchanges, upsizings, and/or balloon dilatations. Successful management of postoperative biliary strictures required an average of 7.5 PBD exchanges, upsizings, and/or balloon dilatations, with median duration of PBD after stricture detection at 5.5 months (patients with benign disease required more interventions than those with malignant neoplasms, 11.7 versus 6.25) [Michael G. House, et al., Incidence and Outcome of Biliary Strictures After Pancreaticoduodenectomy; Ann Surg. 2006 May; 243(5): 571-578] Anastomotic strictures may also be treated with stent placement; however, frequency of and potential risk factors for stent migration remain largely unknown. Malignant strictures, larger diameter stents, and shorter stents were significantly associated with proximal biliary stent migration [Johanson J F, et al., Incidence and risk factors for biliary and pancreatic stent migration. Gastrointest Endosc. 1992 May-June; 38(3):341-6.]. In addition, It has been reported that cholangitis occurs in between 6.7% and 14.3% of postoperative PD [Sanada Y, et al., Recurrent Cholangitis by Biliary Stasis Due to Non-Obstructive Afferent Loop Syndrome After Pylorus-Preserving Pancreatoduodenectomy: Report of a Case; Int Surg. 2014; 99(4): 426-431.]. Most cases of cholangitis originate due to biliary stasis, which is broadly caused by either anastomotic or nonanastomotic stenosis [Sanada, et al, 2014]. Endoscopic retrograde cholangiopancreatography (ERCP), a technique used to study the bile ducts, pancreatic duct, and gallbladder, has been proven to be therapeutically ineffective. Durable resolution of symptoms after surgical revision is unpredictable.

Successful management of postoperative PD strictures includes multiple procedures over a period of nearly 6 months. Metal stent placement is not currently the first line management of PD strictures due to significant risk of stent migration. There is increased risk of recurrent cholangitis secondary to biliary stasis. ERCP and surgical revision are not ideal.

A non-migrating metal stent is designed for short visceral anastomotic strictures and maximized for anti-migration. Current biliary stents on the market have a larger diameter and are designed for long strictures; therefore, the existing stents tend to migrate when placed for anastomotic strictures with short landing zones. A non-migrating visceral metal stent may become first line therapy for treatment of PD strictures.

A non-migrating metal stent can be placed at the same time as initial PTC/PBD placement, significantly decreasing morbidity and hospital visits, as the current standard of care requires multiple procedures over nearly 6 months. A non-migrating metal stent used to treat anastomotic or nonanastomotic stenosis can decrease the incidence of recurrent cholangitis due to biliary stasis, thus decreasing morbidity and potentially mortality.

For example, successful management of postoperative biliary strictures required an average of 7.5 PBD total exchanges, upsizings, and/or balloon dilatations, over an average period of 5.5 months, if this management strategy was successful.

Median duration of PBD after stricture detection was 5.5 months (patients with benign disease required more interventions than those with malignant neoplasms, 11.7 versus 6.25 interventions on average).

A typical frequency for exchanging PBD is every 2-4 weeks, Therefore PBD exchange every 2 weeks, 3 weeks and 4 weeks for a total of 7.5 exchanges would take approximately 3.5 months, 5 months and 7 months respectively to resolve postoperative strictures.

Further, 11.7 versus 6.5 represent PBD average total number of exchanges for postoperative benign versus malignant strictures respectively.

This management strategy is not always successful, and unsuccessful resolution of postoperative strictures are not included in the statistics. Some patients fail this management and therefore have to continue exchanges for the rest of their life.

In addition, each upsizing, and/or balloon dilatation is painful and requires deep sedation or general anesthesia. Routine exchanges for those who fail the current management strategy typically require moderate sedation after the patient gets acclimated to the exchanges.

### Example 2: Ureter/Ileal Conduit Anastomosis Stricture

Ureter/ileal anastomosis strictures occur by same mechanism as biliary enteric anastomosis strictures. Development of scar tissue is secondary to focal ischemia at the anastomosis. The ureter lumen is much smaller, 3-4 mm diameter, entering small bowel. Therefore the non-migrating stent lumen diameter would be smaller and have fewer anchoring hooks (possibly 3 or 4 anchoring hooks, e.g.).

### Example 3: Vascular Indications for Non-Migrating Stents

### Renal Artery Stenosis:

The focal area of stenosis is commonly localized to the ostium, at the takeoff from the aorta. This presents a migration problem for a short stent. If a long stent is often used which sticks far out into the aorta. A normal renal artery is 4-6 mm women, 5-7 mm men. Our stents are usually 5-6 mm to start. Stenosis is usually very short 1-1.5 cm. The landing zone for stent placement is usually short. Central length 1.5-2 cm. Renal artery stenosis is a big problem. We typically use carotid stents off label.

### Celiac Artery Stenosis/Superior Mesenteric Artery Stenosis (Mesenteric Artery Stenosis):

The focal area of stenosis is commonly localized to the ostium, at the takeoff from the aorta. This presents a migration problem for a short stent. Usually this presents in an elderly person with severe abdominal pain and weight loss. This complication presents a real problem for surgeons. (—same as renal artery)

### Renal Vein Stenosis (Nutcracker Syndrome):

Again, migration issues are common due to poor anchoring. Most physicians are reluctant to place a stent. This description typically involves the left renal vein. A normal left renal vein is typically 5.9 cm+/−1.5 cm length and 1.2 cm+/−0.2 cm diameter. However, the gonadal vein shortens the landing zone for stent placement. The bottom line is that a non-migrating stent should be, e.g., 3 cm long, 1.2 cm central, 1.5 cm at ends to over size.

Typically the carotid arteries and renal arteries measure about the same. A superior vena cava (SVC) non-migrating stent should be, e.g., at least 2-2.5 cm diameter, and slightly larger at the ends (additional 2-3 mm, always oversize SVC stents) and 4 cm total length. The bottom line for SVC and great vessels (carotid) is that non-migrating stents having, e.g., 6 mm diameter×2 cm central length may alleviate multiple complications with a single device design.

Another application is for focal stenosis at the take off of the great vessels (common carotid and innominate arteries from the aortic arch). Generally, these examples are vessels with short landing areas for stent placement that present migration problems. The “non-migrating” stent concept described herein can alleviate this concern.

1. A medical device configured to expand or fortify a stricture comprising:
a central conduit section;
at least one anchoring section; and
at least one transitional section positioned between the conduit section and the at least one anchoring section.

2. The device of claim 1, wherein the device comprises two anchoring sections and two transitional sections, wherein each transitional section is positioned between the conduit section and one of the anchoring sections.

3. The device of claim 1, wherein each of the conduit section, the at least one transitional section, and the at least one anchoring section are configured to form a substantially cylindrical structure having an interior lumen and a supportive exterior wall.

4. The device of claim 3, wherein at least a portion of the supportive wall is configured as a mesh having openings.

5. The device of claim 1 wherein at least one section of the device can assume at least two configurations:
(i) a first configuration having a first outer diameter that is substantially constant along the longitudinal axis of the device, and wherein said first outer diameter allows the structure to be inserted in a stricture; and
(ii) a second configuration wherein at least one section of the device has a second outer diameter that is larger than the first outer diameter.

6. The device of claim 1, further comprising anchoring hooks.

7. The device of claim 1, further comprising a living hinge.

8. The device of claim 7, wherein the living hinge comprises an S-hinge, an eyelet hinge, or a trough hinge.

9. The device of claim 5, wherein deployment from a first configuration to a second configuration increases the minimum lumen diameter of the device.

10. The device of claim 5, wherein deployment from a first configuration to a second configuration decreases the length of the device.

11. The device of claim 6, wherein anchoring hooks comprise attachments to the device at different longitudinal points.

12. A medical device for treatment of a stricture, wherein said device can assume at least two configurations, comprising:
a first substantially cylindrical configuration of the device, and
a second configuration of the device wherein at least one region along the cylindrical length of the device is expanded from the initial cylindrical plane.

13. The device of claim 1 configured to comprise a first, second, third, fourth and fifth longitudinal region, wherein:
the first and fifth regions are positioned at the ends of the device and comprise anchoring hooks to function as the anchoring section;
the third region is positioned in the middle of the device to function as the conduit section; and
the second and fourth regions comprise transitional sections, wherein the second region is positioned between the first and third region, and the fourth region is positioned between the third and fifth region.

14. A method for treating a stricture, comprising surgically placing the device of claim 1 into the stricture.

15. The method of claim 14, wherein surgically placing the device comprises deploying the device from a first configuration to a second configuration.

16. A method for using a medical stent device to treat a stricture, comprising:
placing the device into the stricture in a first configuration having a first outer diameter that is relatively constant along the longitudinal axis and that allows the structure to be inserted in the stricture; and
deploying the device into a second configuration having a second outer diameter that is larger than the first outer diameter, so as to secure the device in place.

17. The method of claim 16, wherein deploying the device from a first configuration to a second configuration results in deployment of anchoring hooks positioned at the ends of the device.

18. The method of claim 17, wherein contact between the deployed anchoring hooks and the tissue associated with the stricture provides resistance so as to secure the device in the stricture.

19. The method of claim 16 wherein the device is implanted in a visceral anastomosis stricture to expand or fortify the stricture.

20. The method of claim 16 wherein the device is implanted in a vascular ostium high grade stenosis to expand or fortify the stenosis.

21. The device of claim 12 configured to comprise a first, second, third, fourth and fifth longitudinal region, wherein:
the first and fifth regions are positioned at the ends of the device and comprise anchoring hooks to function as the anchoring section;
the third region is positioned in the middle of the device to function as the conduit section; and
the second and fourth regions comprise transitional sections, wherein the second region is positioned between the first and third region, and the fourth region is positioned between the third and fifth region.

22. The method of claim 15, wherein deploying the device from a first configuration to a second configuration results in deployment of anchoring hooks positioned at the ends of the device.

23. The method of claim 15, wherein contact between the deployed anchoring hooks and the tissue associated with the stricture provides resistance so as to secure the device in the stricture.

24. The method of claim 14 wherein the device is implanted in a visceral anastomosis stricture to expand or fortify the stricture.

25. The method of claim 14 wherein the device is implanted in a vascular ostium high grade stenosis to expand or fortify the stenosis.
